(19) United States
(12) Patent Application Publication
(10) Pub. No.: US 2025/0367281 A1
(43) Pub. Date: Dec. 4, 2025

(54) COW'S MILK PROTEIN REINTRODUCTION KIT

(71) Applicant: Mead Johnson Nutrition Company, Evansville, IN (US)

(72) Inventors: Zafir GAYGADZHIEV, Evansville, IN (US); Ruth April MARTINEZ HAGUE, Slough (GB); Neil SHAH, London (GB)

(21) Appl. No.: 18/876,734

(22) PCT Filed: Jun. 19, 2023
(86) PCT No.: PCT/EP2023/066512

§ 371 Date: Dec. 19, 2024

(30) Foreign Application Priority Data
Jun. 29, 2022 (GB) 2209494.0

(51) Int. Cl. A61K 39/35; A21D 2/26; A61P 37/08

(52) U.S. Cl. CPC A61K 39/35; A21D 2/263; A61P 37/08

(57) ABSTRACT

The present application relates to a cow's milk protein reintroduction kit and a cow's milk protein reintroduction protocol that utilises said cow's milk protein reintroduction kit.

### FIELD OF THE INVENTION

The present application relates to a cow's milk protein reintroduction kit, as well as a cow's milk protein reintroduction protocol. Furthermore, the present application relates to uses of the cow's milk protein reintroduction kit and/or the cow's milk protein reintroduction protocol in: the treatment of cow's milk allergy in a paediatric subject diagnosed with, or believed to be suffering from, cow's milk allergy; and/or, the induction of tolerance to cow's milk in a paediatric subject diagnosed with, or believed to be suffering from, cow's milk allergy. The cow's milk allergy may be non-IgE-mediated cow's milk allergy and/or IgE-mediated cow's milk allergy.

### BACKGROUND

Food allergy is defined as an adverse health effect arising from a specific immune response, which occurs reproducibly on exposure to a given food allergen, with many developed countries seeing a significant rise in the number of children suffering from food allergies over the past three decades (Boyce et al., Nutrition, 2011). Cow's milk allergy (CMA; sometimes referred to as ‘cow's milk protein allergy’ (CMPA)) is a form of food allergy and is an immune-mediated response to one or more proteins in cow's milk that occurs consistently with the ingestion of cow's milk. CMA is one of the most common food allergies in early life, with an estimated prevalence in developed countries ranging from 0.5% to 3% in one year old paediatric subjects (Flom and Sicherer, Nutrients, 2019). In general, the prognosis for CMA is good, with up to 90% of children developing tolerance before three years of age (Venter and Arshad, Pediatr. Clin. North Am., 2011). However, CMA may persist up to school age and beyond, sometimes into adulthood, and may be associated with the development of other allergic diseases, such as asthma, rhinoconjunctivitis, and atopic dermatitis (Sampaio et al., Allergy, 2005), as well as other disease manifestations, such as recurrent abdominal pain (Saps and Bonilla, J. Pediatr., 2011).

According to the European Academy for Allergy and Clinical Immunology (EAACI) and the World Allergy Organisation (WAO) (Johansson et al., J. Allergy Clin. Immunol., 2004), a hypersensitivity reaction to cow's milk can be referred to as CMA if it involves the immune system. Non-allergic cow's milk hypersensitivity, also known as ‘lactose intolerance’, on the other hand, does not involve the immune system. CMA can present with a spectrum of acute or delayed symptoms that can be mild-to-moderate or severe in nature. Symptoms may affect the respiratory, cutaneous, and gastrointestinal systems, or a combination of these systems. CMA can typically be classified as immunoglobulin E (IgE)-mediated CMA or non-IgE-mediated CMA, but there are instances where mixed (i.e. both IgE-mediated and non-IgE-mediated) reactions have been observed.

Infants with IgE-mediated CMA can suffer from immediate-onset IgE-mediated CMA, where the adverse effects appear usually within minutes, up to two hours, following ingestion of cow's milk, or delayed onset non-IgE-mediated CMA where the effects usually develop two or more hours after ingestion of cow's milk (Johansson et al., J. Allergy Clin. Immunol., 2004). It is challenging to define IgE-mediated CMA as ‘mild’, ‘severe’, etc. as external factors often determine the severity of reaction, with anaphylaxis being the most severe presentation (Turner P J, et al., Allergy, 2016). Interestingly, studies have reported that almost 75% of children with IgE-mediated CMA may tolerate baked milk containing foods like muffins, cakes, breads, and waffles (Nowak-Wegrzyn et al., J. Allergy Clin. Immunol., 2008). Diagnosis of IgE-mediated CMA is usually done via a combination of clinical history and further testing i.e. either a skin prick test or a blood test, for specific IgE antibodies to the suspected food allergen.

The spectrum of non-IgE-mediated CMA is broad and encompasses symptoms that range in severity from rectal bleeding in milk protein-induced proctocolitis (mild-to-moderate non-IgE-mediated CMA) to the acute or chronic vomiting and collapse that can be seen in food protein-induced enterocolitis syndrome (FPIES; severe non-IgE-mediated CMA). In the UK, it has been shown that the majority of infants presenting with suspected CMA fall into a ‘mild-to-moderate’ clinical expression of non-IgE-mediated CMA (Sladkevicius et al., J. Med. Econ., 2010). Diagnosis of mild-to-moderate non-IgE-mediated CMA involves the trial elimination of the cow's milk(-containing food products) for a two-to-four-week period, with a planned and intentional reintroduction of cow's milk(-containing food products). If clinical improvement is seen during the elimination diet and is then followed by subsequent relapse coincident with reintroduction of cow's milk, mild-to-moderate non-IgE-mediated CMA is confirmed. This elimination-reintroduction sequence is the only way of reliably diagnosing gastrointestinal manifestations of non-IgE-mediated CMA in infants, such as infantile allergic proctocolitis, mild-to-moderate allergic enteropathy, and cow's milk-induced gastro-oesophageal reflux or constipation, as there is no skin test or blood test for non-IgE-mediated CMA.

Overcoming CMA, or at least developing a level of tolerance to cow's milk, can be particularly important for a paediatric subject as cow's milk formula and/or cow's milk-containing foods play an important role in the nutritional intake of children, particularly in early infancy. Further, suffering from CMA has a negative effect on the paediatric subjects, as well as the wider family's, quality of life.

At the present moment, there are no clinically-established and commercially available IgE-mediated CMA treatment methods. Oral immunotherapy is a potential method of treating IgE-mediated CMA, but this is still at the clinical trial stage and is not without risk. Therefore, paediatric subjects with IgE-mediated CMA have to rely on management by individualised avoidance, rather than treatment, of their IgE-mediated CMA. Management of IgE-mediated CMA typically involves elimination of cow's milk from the subject's diet, ensuring that the subject is always in close proximity to an epinephrine autoinjector, etc.

Current non-IgE-mediated CMA treatment methods typically involve a period of elimination, followed by the reintroduction of foods containing cow's milk, with the foods increasing in amount, and varying in form, of cow's milk protein. A drawback of the current non-IgE-mediated CMA treatment methods is that the onus is typically on the parent/caregiver to prepare the various different cow's milk-containing foods, which can often require precision in the amount of milk added to the particular food(s), precision in the temperature and/or time of cooking, and the incorporation of numerous ingredients, in addition to milk, in order to provide a complex cow's milk-containing food. It is also difficult for the parent/caregiver to know which cow's milk-containing foods should be given to the paediatric subject, when, and how much of said cow's milk-containing food(s). Dealing with a paediatric subject diagnosed with, or believed to be suffering from, CMA is stressful enough for the parent/caregiver, without this added complexity, burden, and stress.

Accordingly, there exists a need for a mechanism for reintroducing cow's milk into the diet of a paediatric subject diagnosed with, or believed to be suffering from, cow's milk allergy that is simple and convenient for the parent/caregiver to employ, and yet still effective.

### SUMMARY OF INVENTION

In a first aspect, there is provided a cow's milk protein reintroduction kit comprising:
at least one first discrete unit comprising a composition comprising 0.01 to 0.08 g of cow's milk protein;
at least one second discrete unit comprising a composition comprising 0.25 to 0.54 g of cow's milk protein; and,
at least one third discrete unit comprising a composition comprising 0.55 to 2 g of cow's milk protein.

Preferably, the first discrete unit, the second discrete unit, and/or the third discrete unit further comprises a carbohydrate source. More preferably, the carbohydrate source is maltodextrin, lactose, corn syrup solids, starch, permeate, glucose solids, or any combination thereof.

Preferably, the 0.01 to 0.08 g of cow's milk protein of the first discrete unit comprises 5 to 55 micrograms (μg) of Bos d 5, the 0.25 to 0.54 g of cow's milk protein of the second discrete unit comprises 170 to 400 μg of Bos d 5, and the 0.55 to 2 g of cow's milk protein of the third discrete unit comprises at least 1250 μg of Bos d 5.

Preferably, the at least one first discrete unit comprises a composition comprising 0.01 to 0.07 g of cow's milk protein. More preferably, the at least one first discrete unit comprises a composition comprising 0.01 to 0.05 g of cow's milk protein.

Preferably, the at least one first discrete unit comprises two to one hundred first discrete units.

Preferably, the at least one first discrete unit comprises cow's milk protein provided by a milk source, wherein the milk source comprises whole milk, whole milk powder, whole milk isolates, lactose-free whole milk, lactose-free whole milk powder, lactose-free whole milk isolates, skimmed (non-fat) milk, skimmed (non-fat) milk powder, skimmed (non-fat) milk isolates, lactose-free skimmed (non-fat) milk, lactose-free skimmed (non-fat) milk powder, lactose-free skimmed (non-fat) milk isolates, buttermilk or any combination thereof.

Preferably, the at least one second discrete unit comprises a composition comprising 0.25 to 0.5 g of cow's milk protein. More preferably, the at least one second discrete unit comprises a composition comprising 0.25 to 0.45 g of cow's milk protein.

Preferably, the at least one second discrete unit comprises two to one hundred second discrete units.

Preferably, the at least one second discrete unit comprises cow's milk protein provided by a milk source, wherein the milk source comprises whole milk, whole milk powder, whole milk isolates, lactose-free whole milk, lactose-free whole milk powder, lactose-free whole milk isolates, skimmed (non-fat) milk, skimmed (non-fat) milk powder, skimmed (non-fat) milk isolates, lactose-free skimmed (non-fat) milk, lactose-free skimmed (non-fat) milk powder, lactose-free skimmed (non-fat) milk isolates, buttermilk, or any combination thereof.

Preferably, the at least one third discrete unit comprises a composition comprising 0.55 to 1.5 g of cow's milk protein. More preferably, the at least one first discrete unit comprises a composition comprising 0.55 to 1 g of cow's milk protein.

Preferably, the at least one third discrete unit comprises cow's milk protein provided by a milk source, wherein the milk source comprises whole milk, whole milk powder, whole milk isolates, lactose-free whole milk, lactose-free whole milk powder, lactose-free whole milk isolates, skimmed (non-fat) milk, skimmed (non-fat) milk powder, skimmed (non-fat) milk isolates, lactose-free skimmed (non-fat) milk, lactose-free skimmed (non-fat) milk powder, lactose-free skimmed (non-fat) milk isolates, buttermilk or any combination thereof.

Preferably, the at least one third discrete unit comprises two to one hundred third discrete units.

Preferably, the composition of the first discrete unit, the composition of the second discrete unit, and/or the composition of the third discrete unit is a supplement or a nutritional composition.

Preferably, the composition of the first discrete unit, the composition of the second discrete unit, and/or the composition of the third discrete unit is a synthetic composition.

In a second aspect, there is provided the cow's milk protein reintroduction kit of the first aspect for use in the treatment of cow's milk allergy in a paediatric subject diagnosed with, or believed to be suffering from, cow's milk allergy.

Preferably, the cow's milk allergy is non-IgE-mediated cow's milk allergy and/or IgE-mediated cow's milk allergy. More preferably, when the cow's milk allergy is non-IgE-mediated cow's milk allergy, the non-IgE-mediated cow's milk allergy is mild-to-moderate (i.e. non-severe) non-IgE-mediated cow's milk allergy.

In a third aspect, there is provided the cow's milk protein reintroduction kit of the first aspect for use in the induction of tolerance to cow's milk in a paediatric subject diagnosed with, or believed to be suffering from, cow's milk allergy.

Preferably, the cow's milk allergy is non-IgE-mediated cow's milk allergy and/or IgE-mediated cow's milk allergy. More preferably, when the cow's milk allergy is non-IgE-mediated cow's milk allergy, the non-IgE-mediated cow's milk allergy is mild-to-moderate (i.e. non-severe) non-IgE-mediated cow's milk allergy.

In a fourth aspect, there is provided a cow's milk protein reintroduction protocol for use in the treatment of cow's milk allergy in a paediatric subject diagnosed with, or believed to be suffering from, cow's milk allergy, comprising the steps of:
Providing the paediatric subject with one first discrete unit for at least one day, wherein the first discrete unit comprises a composition comprising 0.01 to 0.08 g of cow's milk protein;
Providing the paediatric subject with two first discrete units for at least one day;
Providing the paediatric subject with three first discrete units for at least one day;
Providing the paediatric subject with one second discrete unit for at least one day, wherein the second discrete unit comprises a composition comprising 0.25 to 0.54 g of cow's milk protein;
Providing the paediatric subject with two second discrete units for at least one day;
Providing the paediatric subject with three second discrete units for at least one day;
Providing the paediatric subject with one third discrete unit for at least one day, wherein the third discrete unit comprises a composition comprising 0.55 to 2 g of cow's milk protein;
Providing the paediatric subject with two third discrete units for at least one day; and,
Providing the paediatric subject with three third discrete units for at least one day.

Preferably, the 0.01 to 0.08 g of cow's milk protein of the first discrete unit comprises 5 to 55 micrograms (μg) of Bos d 5 the 0.25 to 0.54 g of cow's milk protein of the second discrete unit comprises 170 to 400 μg of Bos d 5, and the 0.55 to 2 g of cow's milk protein of the third discrete unit comprises at least 1250 μg of Bos d 5.

Preferably, the cow's milk allergy is non-IgE-mediated cow's milk allergy and/or IgE-mediated cow's milk allergy. More preferably, when the cow's milk allergy is non-IgE-mediated cow's milk allergy, the non-IgE-mediated cow's milk allergy is mild-to-moderate (i.e. non-severe) non-IgE-mediated cow's milk allergy.

In a fifth aspect, there is provided a cow's milk protein reintroduction protocol for use in the induction of tolerance to cow's milk in a paediatric subject diagnosed with, or believed to be suffering from, cow's milk allergy, comprising the steps of:
Providing the paediatric subject with one first discrete unit for at least one day, wherein the first discrete unit comprises a composition comprising 0.01 to 0.08 g of cow's milk protein;
Providing the paediatric subject with two first discrete units for at least one day;
Providing the paediatric subject with three first discrete units for at least one day;
Providing the paediatric subject with one second discrete unit for at least one day, wherein the second discrete unit comprises a composition comprising 0.25 to 0.54 g of cow's milk protein;
Providing the paediatric subject with two second discrete units for at least one day; and,
Providing the paediatric subject with three second discrete units for at least one day;
Providing the paediatric subject with one third discrete unit for at least one day, wherein the third discrete unit comprises a composition comprising 0.55 to 2 g of cow's milk protein;
Providing the paediatric subject with two third discrete units for at least one day; and,
Providing the paediatric subject with three third discrete units for at least one day.

Preferably, the 0.01 to 0.08 g of cow's milk protein of the first discrete unit comprises 5 to 55 micrograms (μg) of Bos d 5 the 0.25 to 0.54 g of cow's milk protein of the second discrete unit comprises 170 to 400 μg of Bos d 5, and the 0.55 to 2 g of cow's milk protein of the third discrete unit comprises at least 1250 μg of Bos d 5.

Preferably, the cow's milk allergy is non-IgE-mediated cow's milk allergy and/or IgE-mediated cow's milk allergy. More preferably, when the cow's milk allergy is non-IgE-mediated cow's milk allergy, the non-IgE-mediated cow's milk allergy is mild-to-moderate (i.e. non-severe) non-IgE-mediated cow's milk allergy.

In a sixth aspect, there is provided a cow's milk protein reintroduction kit comprising:
at least one first discrete unit comprising a composition comprising 5 to 55 micrograms (μg) of Bos d 5;
at least one second discrete unit comprising a composition comprising 170 to 400 μg of Bos d 5; and,
at least one third discrete unit comprising a composition comprising at least 1250 μg of Bos d 5.

Preferably, the first discrete unit, the second discrete unit, and/or the third discrete unit further comprises a carbohydrate source. More preferably, the carbohydrate source is maltodextrin, lactose, corn syrup solids, starch, permeate, glucose solids, or any combination thereof.

Preferably, the first discrete unit comprises a composition comprising 15 to 55 μg of Bos d 5.

Preferably, the at least one first discrete unit comprises two to one hundred first discrete units.

Preferably, the at least one first discrete unit comprises cow's milk protein provided by a milk source, wherein the milk source comprises whole milk, whole milk powder, whole milk isolates, lactose-free whole milk, lactose-free whole milk powder, lactose-free whole milk isolates, skimmed milk, skimmed milk powder, skimmed milk isolates, lactose-free skimmed milk, lactose-free skimmed (non-fat) milk powder, lactose-free skimmed milk isolates, buttermilk or any combination thereof.

Preferably, the second discrete unit comprises a composition comprising 200 to 350 μg of Bos d 5.

Preferably, the at least one second discrete unit comprises two to one hundred second discrete units.

Preferably, the at least one second discrete unit comprises cow's milk protein provided by a milk source, wherein the milk source comprises whole milk, whole milk powder, whole milk isolates, lactose-free whole milk, lactose-free whole milk powder, lactose-free whole milk isolates, skimmed milk, skimmed milk powder, skimmed milk isolates, lactose-free skimmed milk, lactose-free skimmed (non-fat) milk powder, lactose-free skimmed milk isolates, buttermilk, or any combination thereof.

Preferably, the at least one third discrete unit comprises a composition comprising 200 to 350 μg of Bos d 5.

Preferably, the at least one third discrete unit comprises cow's milk protein provided by a milk source, wherein the milk source comprises whole milk, whole milk powder, whole milk isolates, lactose-free whole milk, lactose-free whole milk powder, lactose-free whole milk isolates, skimmed milk, skimmed milk powder, skimmed milk isolates, lactose-free skimmed milk, lactose-free skimmed milk powder, lactose-free skimmed milk isolates, buttermilk or any combination thereof.

Preferably, the at least one third discrete unit comprises two to one hundred third discrete units.

Preferably, the composition of the first discrete unit, the composition of the second discrete unit, and/or the composition of the third discrete unit is a supplement or a nutritional composition.

Preferably, the composition of the first discrete unit, the composition of the second discrete unit, and/or the composition of the third discrete unit is a synthetic composition.

In a seventh aspect, there is provided the cow's milk protein reintroduction kit of the sixth aspect for use in the treatment of cow's milk allergy in a paediatric subject diagnosed with, or believed to be suffering from, cow's milk allergy.

Preferably, the cow's milk allergy is non-IgE-mediated cow's milk allergy and/or IgE-mediated cow's milk allergy. More preferably, when the cow's milk allergy is non-IgE-mediated cow's milk allergy, the non-IgE-mediated cow's milk allergy is mild-to-moderate (i.e. non-severe) non-IgE-mediated cow's milk allergy.

In an eighth aspect, there is provided the cow's milk protein reintroduction kit of the sixth aspect for use in the induction of tolerance to cow's milk in a paediatric subject diagnosed with, or believed to be suffering from, cow's milk allergy.

Preferably, the cow's milk allergy is non-IgE-mediated cow's milk allergy and/or IgE-mediated cow's milk allergy. More preferably, when the cow's milk allergy is non-IgE-mediated cow's milk allergy, the non-IgE-mediated cow's milk allergy is mild-to-moderate (i.e. non-severe) non-IgE-mediated cow's milk allergy.

In a ninth aspect, there is provided a cow's milk protein reintroduction protocol for use in the treatment of cow's milk allergy in a paediatric subject diagnosed with, or believed to be suffering from, cow's milk allergy, comprising the steps of:
Providing the paediatric subject with one first discrete unit for at least one day, wherein the first discrete unit comprises a composition comprising 5 to 55 μg of Bos d 5;
Providing the paediatric subject with two first discrete units for at least one day;
Providing the paediatric subject with three first discrete units for at least one day;
Providing the paediatric subject with one second discrete unit for at least one day, wherein the second discrete unit comprises a composition comprising 170 to 400 μg of Bos d 5;
Providing the paediatric subject with two second discrete units for at least one day;
Providing the paediatric subject with three second discrete units for at least one day;
Providing the paediatric subject with one third discrete unit for at least one day, wherein the third discrete unit comprises a composition comprising at least 1250 μg of Bos d 5;
Providing the paediatric subject with two third discrete units for at least one day; and,
Providing the paediatric subject with three third discrete units for at least one day.

Preferably, the cow's milk allergy is non-IgE-mediated cow's milk allergy and/or IgE-mediated cow's milk allergy. More preferably, when the cow's milk allergy is non-IgE-mediated cow's milk allergy, the non-IgE-mediated cow's milk allergy is mild-to-moderate (i.e. non-severe) non-IgE-mediated cow's milk allergy.

In a tenth aspect, there is provided a cow's milk protein reintroduction protocol for use in the induction of tolerance to cow's milk in a paediatric subject diagnosed with, or believed to be suffering from, cow's milk allergy, comprising the steps of:
Providing the paediatric subject with one first discrete unit for at least one day, wherein the first discrete unit comprises a composition comprising 5 to 55 μg of Bos d 5;
Providing the paediatric subject with two first discrete units for at least one day;
Providing the paediatric subject with three first discrete units for at least one day;
Providing the paediatric subject with one second discrete unit for at least one day, wherein the second discrete unit comprises a composition comprising 170 to 400 μg of Bos d 5;
Providing the paediatric subject with two second discrete units for at least one day;
Providing the paediatric subject with three second discrete units for at least one day;
Providing the paediatric subject with one third discrete unit for at least one day, wherein the third discrete unit comprises a composition comprising at least 1250 μg of Bos d 5;
Providing the paediatric subject with two third discrete units for at least one day; and,
Providing the paediatric subject with three third discrete units for at least one day.

Preferably, the cow's milk allergy is mild-to-moderate (i.e. non-severe) non-IgE-mediated cow's milk allergy and/or IgE-mediated cow's milk allergy. More preferably, when the cow's milk allergy is non-IgE-mediated cow's milk allergy, the non-IgE-mediated cow's milk allergy is mild-to-moderate (i.e. non-severe) non-IgE-mediated cow's milk allergy.

### Definitions

“Milk”, in terms of the present disclosure, means a substance that has been drawn or extracted from the mammary gland of a mammal or any non-dairy, vegan-friendly, and/or plant-based alternative milk. Examples of milks are bovine milk, caprine milk, ovine milk, coconut milk, soy milk, almond milk, oat milk, hazelnut milk, pea milk, potato milk, quinoa milk, or any combination thereof.

“Cow's milk protein”, in terms of the present disclosure, refers to a protein found in cow's milk, or a protein with equivalent allergenicity to a protein found in cow's milk, a protein with a high level of sequence homology to that of a protein found in cow's milk, and/or a protein with a high level of sequence similarity to that of a protein found in cow's milk, found in one or more other milks. The expression “a high level of sequence homology”, in terms of the present disclosure, means a sequence homology of at least 50%. The expression “a high level of sequence similarity”, in terms of the present disclosure, means a sequence similarity of at least 50%.

“Bos d 5” refers to β-lactoglobulin, a protein found in certain milks e.g. cow's milk, sheep's milk, etc.

“Nutritional composition” means a substance or composition that satisfies at least a portion of a subject's nutrient requirements. “Nutritional composition(s)” may be provided in the form of a powder, a gel, a suspension, a paste, a solid, a gummy, a liquid, a liquid concentrate, a reconstitutable powder, a reconstituted solution, or a ready-to-use product. “Nutritional composition(s)” may refer to a nutritional supplement, a children's nutritional product, an infant formula, a human milk fortifier, a follow-up formula, a young child milk, or any other composition designed for a paediatric subject.

“Supplement”, in terms of the present disclosure, refers to a substance or composition that does not necessarily satisfy at least a portion of a subject's nutrient requirements. “Supplement(s)” may be provided in the form of a powder, a gel, a suspension, a paste, a solid, a gummy, a liquid, a liquid concentrate, a reconstitutable powder, a reconstituted solution, or a ready-to-use product.

“Reconstituted solution”, in terms of the present disclosure, means the solution prepared when a diluent (e.g. water, saline, etc.) is added to an ingredient (e.g. a powder, a solution, a gel, a suspension, a paste, a solid, a liquid, a liquid concentrate, etc.).

“Ready-to-use product”, in terms of the present disclosure, refers to a sterile liquid product that is provided in a form that is suitable for immediate ingestion by a subject.

The term “synthetic” when applied to a composition, a nutritional composition, or a mixture means a composition, nutritional composition, or mixture obtained by biological and/or chemical means, which can be chemically identical to the mixture naturally occurring in mammalian milks. A composition, nutritional composition, or mixture is said to be “synthetic” if at least one of its components is obtained by biological (e.g. enzymatic) and/or chemical means.

“Paediatric subject” means a human under 18 years of age. The term “paediatric subject” may refer to preterm infants, full-term infants, and/or children, as described below. A paediatric subject may be a human subject that is between birth and 8 years old. In another aspect, “paediatric subject” refers to a human subject between 1 and 6 years of age. Alternatively, “paediatric subject” refers to a human subject between 6 and 12 years of age.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes full-term infants, preterm infants, low birth weight infants, very low birth weight infants, and extremely low birth weight infants. “Preterm” means an infant born before the end of the 37th week of gestation. “Full-term” means an infant born after the end of the 37th week of gestation.

“Child” means a subject ranging from 12 months to 13 years of age. A child may be a subject between the ages of 1 and 12 years old. In another aspect, the terms “children” or “child” may refer to subjects that are between 1 and about 6 years old. Alternatively, the terms “children” or “child” may refer to subjects that are between about 7 and about 12 years old. The term “young child” means a subject ranging from 1 year to 3 years of age.

“Infant formula” means a composition that satisfies at least a portion of the nutrient requirements of an infant.

“Follow-up formula” means a composition that satisfies at least a portion of the nutrient requirements of an infant from the 6th month onwards, and for young children from 1 to 3 years of age.

“Young child milk”, in terms of the present disclosure, means a fortified milk-based beverage intended for children over one year of age (typically from one to six years of age). Young child milks are designed with the intent to serve as a complement to a diverse diet, to provide additional insurance that a child achieves continual, daily intake of all essential vitamins and minerals, macronutrients plus additional functional dietary components, such as non-essential nutrients that have purported health-promoting properties.

“Milk challenge” refers to the deliberate milk exposure, for the explicit purposes of a diagnosis of CMA (usually after a two-to-four-week period of avoidance) and can be done at home or in hospital. For an IgE-mediated cow's milk allergic infant, this is usually done in hospital.

“Milk reintroduction” and “milk rechallenge”, are used interchangeably in the present disclosure and, in terms of the present disclosure, refer to the gradual reintroduction of cow's milk protein, after an extended period of avoidance, for the explicit purposes of developing tolerance to cow's milk. For a non-IgE-mediated cow's milk allergic infant, this can usually be done at home.

“Discrete unit”, in terms of the present disclosure, refers to the manner in which a specific, consistent amount of a composition is provided in an individual, distinct form. By way of example, if the cow's milk reintroduction kit comprises more than one e.g. “first discrete unit”, each individual first discrete unit comprises the same amount of the same composition. Further, a first discrete unit comprises a different composition to that of a second discrete unit, but may contain the same total amount (i.e. weight, volume, etc.) of composition, despite comprising different compositions. The discrete unit may be in the form of a sachet, a packet, a stick, a stick packet, a package, a parcel, a tub, a container, a capsule, or any combination thereof.

The expression “treatment of cow's milk allergy”, in terms of the present disclosure, describes the situation wherein a subject shows a reduced number and/or severity of clinically-relevant symptoms or ill effects (i.e. those detailed in Table 1; see ‘Examples’ section) after ingestion of cow's milk and/or a cow's milk containing product. Treatment of cow's milk allergy is therefore an intervention with the ultimate aim of acquiring tolerance to cow's milk and/or a cow's milk containing product. When the cow's milk allergy is non-IgE-mediated CMA, particularly mild-to-moderate (i.e. non-severe) non-IgE-mediated CMA, treatment of cow's milk allergy may be determined by initiating a milk rechallenge (i.e. milk reintroduction) and observing a decrease in the number and/or severity of clinically-relevant symptoms as the paediatric subject progresses through the milk rechallenge (see ‘Experimental Procedure’ section, Study 2). When the cow's milk allergy is IgE-mediated CMA, treatment of cow's milk allergy may be determined by initiating a milk rechallenge (i.e. milk reintroduction) and: (i) observing a decrease in the number and/or severity of clinically-relevant symptoms as the paediatric subject progresses through the milk rechallenge; (ii) measuring the immunoglobulin E (IgE) levels in sera; and/or, (iii) measuring the immunoglobulin G4 (IgG4) levels in sera (see ‘Experimental Procedure’ section, Study 3).

The expression “induction of tolerance to cow's milk”, in terms of the present disclosure, describes the situation wherein a subject shows no clinically-relevant symptoms or ill effects (i.e. those detailed in Table 1; see ‘Examples’ section) after ingestion of cow's milk and/or a cow's milk containing product. Tolerance is the permanent resolution of cow's milk allergy and is the optimal goal of treatment. When the cow's milk allergy is non-IgE-mediated CMA, particularly mild-to-moderate (i.e. non-severe) non-IgE-mediated CMA, induction of tolerance to cow's milk may be determined by initiating a milk rechallenge (i.e. milk reintroduction) and observing no clinically-relevant symptoms at the completion of the milk rechallenge (see ‘Experimental Procedure’ section, Study 2). When the cow's milk allergy is IgE-mediated CMA, induction of tolerance to cow's milk may be determined by initiating a milk rechallenge (i.e. milk reintroduction) and: (i) observing no clinically-relevant symptoms at the completion of the milk rechallenge; (ii) measuring the immunoglobulin E (IgE) levels in sera; and/or, (iii) measuring the immunoglobulin G4 (IgG4) levels in sera (see ‘Experimental Procedure’ section, Study 3).

The term “substantially free” means containing less than a functional amount of the specified component, typically less than 0.1% by weight, and includes 0% by weight of the specified ingredient.

All percentages, parts, and ratios as used herein are detailed by weight of the total composition, unless otherwise specified. All amounts specified as administered “per day” may be delivered in a single unit dose, in a single serving, or in two or more doses or servings administered over the course of a 24-hour period.

All references to singular characteristics or limitations in the present disclosure shall include the corresponding plural characteristic or limitation, and vice versa, unless otherwise specified or clearly implied to the contrary, by the context in which the reference is made.

The compositions of the present disclosure can comprise, consist of, or consist essentially of any of the components described herein, as well as including any additional useful component.

### DETAILED DESCRIPTION

The present invention provides a novel mechanism for cow's milk protein reintroduction for paediatric subjects diagnosed with, or believed to be suffering from, non-IgE-mediated cow's milk allergy or IgE-mediated cow's milk allergy, via the provision of a cow's milk protein reintroduction kit and/or a cow's milk protein reintroduction protocol.

The inventors have appreciated the need for a simple, convenient, and effective mechanism of reintroducing cow's milk into the diet of a paediatric subject diagnosed with, or believed to be suffering from, cow's milk allergy, and have devised the cow's milk protein reintroduction kit and cow's milk protein reintroduction protocol described subsequently. A paediatric subject that is able to tolerate cow's milk has access to a wider variety of important paediatric nutritional products, than a paediatric subject with CMA. Thus, it is much easier for a cow's milk tolerant paediatric subject to obtain their required daily nutritional intake than it is for a paediatric subject with CMA. This has a pronounced effect on not only the paediatric subject's quality of life, but also that of the parents'/caregivers' lives too. It is believed that this earlier introduction of potential allergens may reduce the likelihood of the paediatric subject developing other (food-related) allergies later in life.

In a first aspect, there is provided a cow's milk protein reintroduction kit comprising:
at least one first discrete unit comprising a composition comprising 0.01 to 0.08 g of cow's milk protein;
at least one second discrete unit comprising a composition comprising 0.25 to 0.54 g of cow's milk protein; and,
at least one third discrete unit comprising a composition comprising 0.55 to 2 g of cow's milk protein.

The cow's milk protein reintroduction kit may be used as detailed in Example 1 in the ‘Examples’ section below.

The cow's milk protein of the composition of the first discrete unit, the composition of the second discrete unit, and/or the composition of the third discrete unit may be provided by a milk source. The milk source may comprise whole milk, whole milk powder, lactose-free whole milk, lactose-free whole milk powder, whole milk concentrates, whole milk isolates, lactose-free whole milk isolates, whole milk solids, semi-skimmed (reduced fat) milk, semi-skimmed (reduced fat) milk powder, lactose-free semi-skimmed (reduced fat) milk, lactose-free semi-skimmed (reduced fat) milk powder, semi-skimmed (reduced fat) milk concentrates, semi-skimmed (reduced fat) milk isolates, lactose-free semi-skimmed (reduced fat) milk isolates, semi-skimmed (reduced fat) milk solids, skimmed (non-fat) milk, skimmed (non-fat) milk powder, lactose-free skimmed (non-fat) milk, lactose-free skimmed (non-fat) milk powder, skimmed (non-fat) milk concentrates, skimmed (non-fat) milk isolates, lactose-free skimmed (non-fat) milk isolates, skimmed (non-fat) milk solids, buttermilk, or any combination thereof. Preferably, the milk source is provided by whole milk, whole milk powder, whole milk isolates, lactose-free whole milk, lactose-free whole milk powder, lactose-free whole milk isolates, skimmed (non-fat) milk, skimmed (non-fat) milk powder, skimmed (non-fat) milk isolates, lactose-free skimmed (non-fat) milk, lactose-free skimmed (non-fat) milk powder, lactose-free skimmed (non-fat) milk isolates, buttermilk, or any combination thereof. The milk source of the compositions of the first discrete unit, the second discrete unit, and the third discrete unit may be provided by the same milk source. Alternatively, the milk source of the compositions may be provided by the same milk source for two out of the three compositions of the first discrete unit, the second discrete unit, and the third discrete unit. Alternatively, the milk source of the compositions of the first discrete unit, the second discrete unit, and the third discrete unit may each be provided by a different milk source. The milk source may be derived from bovine milk, caprine milk, ovine milk, or any combination thereof. Preferably, the milk source is derived from bovine milk.

The cow's milk protein may be provided by an alternative milk source. The alternative milk source may be any non-dairy, vegan-friendly, and/or plant-based alternative milk source. The alternative milk source may comprise coconut milk, soy milk, almond milk, oat milk, hazelnut milk, pea milk, potato milk, cashew milk, quinoa milk, or any combination thereof.

The cow's milk protein of the composition of the first discrete unit, the composition of the second discrete unit, and/or the composition of the third discrete unit may be provided by a milk source and/or an alternative milk source that has been subjected to one or more additional treatments, such as filtration, microfiltration, precipitation, hydrolysis, extraction, crystallisation, fractionation, centrifugation, distillation, chromatography, diafiltration, enzyme digestion, or any other suitable treatment process known in the art, to yield a treated milk source and/or a treated alternative milk source. The cow's milk protein may therefore be provided by a treated milk source and/or a treated alternative milk source comprising a partial hydrolysate, an extensively hydrolysed milk source hydrolysate, a milk source co-precipitate, an alternative milk source partial hydrolysate, an extensively hydrolysed alternative milk source hydrolysate, an alternative source co-precipitate, a whey protein concentrate, a whey protein isolate, one or more whey protein (such as alpha-lactalbumin, beta-lactoglobulin, or any combination thereof), a casein concentrate, a casein isolate, one or more casein protein (such as casein glycomacropeptide), a caseinate, a casein protein formed from amino acids, or any combination thereof.

The composition of the first discrete unit may comprise 0.01 to 0.07 g of cow's milk protein. Preferably, the composition of the first discrete unit comprises 0.01 to 0.05 g of cow's milk protein.

The at least one first discrete unit may comprise two to one hundred first discrete units, two to fifty first discrete units, two to thirty first discrete units, or six to twenty-four first discrete units.

The composition of the second discrete unit may comprise 0.25 to 0.5 g of cow's milk protein. Preferably, the composition of the second discrete unit comprises 0.45 to 0.6 g of cow's milk protein.

The at least one second discrete unit may comprise two to one hundred second discrete units, two to fifty second discrete units, two to thirty second discrete units, or six to twenty-four second discrete units.

The composition of the third discrete unit may comprise 0.55 to 1.5 g of cow's milk protein. Preferably, the composition of the third discrete unit comprises 0.55 to 1 g of cow's milk protein.

The at least one third discrete unit may comprise two to one hundred third discrete units, two to fifty third discrete units, two to thirty third discrete units, or six to twenty-four third discrete units.

The composition of the first discrete unit, the composition of the second discrete unit, and the composition of the third discrete unit may each be subjected to different processing conditions during production (i.e. temperature, hold time, etc.), so as to yield compositions with different levels of allergenicity to each other.

The first discrete unit, the second discrete unit, and the third discrete unit may be in the form of a sachet, a packet, a stick, a stick packet, a package, a parcel, a tub, a container, a capsule, or any combination thereof. The first discrete unit, the second discrete unit, and the third discrete unit may each be provided in the same form. Alternatively, two out of the three of the first discrete unit, the second discrete unit, and the third discrete unit may be provided in the same form. Alternatively, the first discrete unit, the second discrete unit, and the third discrete unit may each be provided in a different form.

The proteins in cow's milk are about 80% casein proteins and 20% whey proteins, with the whey proteins consisting mainly of β-lactoglobulin (also known as Bos d 5), α-lactalbumin, and serum albumin. Human milk lacks a Bos d 5 homologue, thus Bos d 5 has long been considered one of the most important mammalian milk allergens e.g. cow's milk, sheep's milk, etc. (Roth-Walter et al., PLOS One, 2014; Fiocchi et al., The World Allergy Organ. J., 2010).

The inventors hypothesize that a cow's milk protein reintroduction kit based on the amount of Bos d 5 in common cow's milk-containing foods will provide an effective mechanism of reintroducing cow's milk into the diet of a paediatric subject diagnosed with, or believed to be suffering from, cow's milk allergy. The inventors have therefore determined the amount of Bos d 5 in common cow's milk-containing foods (a baked cookie, a baked muffin, and liquid skimmed (non-fat) milk) and have devised the cow's milk protein reintroduction kit and cow's milk protein reintroduction protocol, accordingly. The amount of Bos d 5 in common cow's milk-containing foods may be quantified by the method detailed in Study 1 of the ‘Experimental Procedure’ section below, or any suitable method for determining Bos d 5 levels known in the art.

The 0.01 to 0.08 g of cow's milk protein of the composition of the first discrete unit may comprise 5 to 55 micrograms (μg) of Bos d 5, 10 to 55 μg of Bos d 5, 10 to 50 μg of Bos d 5, 15 to 50 μg of Bos d 5, 15 to 45 μg of Bos d 5, 20 to 45 μg of Bos d 5, or 25 to 45 μg of Bos d 5.

The 0.25 to 0.54 g of cow's milk protein of the composition of the second discrete unit may comprise 170 to 400 μg of Bos d 5, 180 to 400 μg of Bos d 5, 190 to 400 μg of Bos d 5, 200 to 400 μg of Bos d 5, 200 to 350 μg of Bos d 5, 210 to 350 μg of Bos d 5, 210 to 300 μg of Bos d 5, or 220 to 280 μg of Bos d 5.

The 0.55 to 2 g of cow's milk protein of the composition of the third discrete unit may comprise at least 1250 μg of Bos d 5, 1250 μg to 75 milligrams (mg) of Bos d 5, 1250 μg to 70 mg of Bos d 5, 1500 μg to 70 mg of Bos d 5, 1500 μg to 60 mg of Bos d 5, 2000 μg to 60 mg of Bos d 5, or 2000 μg to 50 mg of Bos d 5.

The composition of the first discrete unit, the composition of the second discrete unit, and/or the composition of the third discrete unit may comprise a carbohydrate source. It is believed that the combination of cow's milk protein and a carbohydrate source could result in a reduction in cow's milk protein allergenicity and thus, be beneficial in the process of reintroducing cow's milk into the diet of a paediatric subject. The carbohydrate source may comprise maltodextrin, lactose, corn syrup solids, starch, dextrins, permeate, glucose solids, or any combination thereof. Preferably, the carbohydrate source is maltodextrin, lactose, or a combination thereof.

The carbohydrate source may be present in the composition of the first discrete unit, the composition of the second discrete unit, and/or the composition of the third discrete unit in an amount of 0.01 to 10 g. Preferably, the carbohydrate source is present in in the composition of the first discrete unit, the composition of the second discrete unit, and/or the composition of the third discrete unit in an amount of 0.05 to 5 g. More preferably, the carbohydrate source is present in the composition of the first discrete unit, the composition of the second discrete unit, and/or the composition of the third discrete unit in an amount of 0.1 to 2 g.

The composition of the first discrete unit, the composition of the second discrete unit, and/or the composition of the third discrete unit may comprise a free-flowing agent. The free-flowing agent aids with the flowability of the composition of the first discrete unit, the composition of the second discrete unit, and/or the composition of the third discrete unit. The free-flowing agent may comprise tricalcium phosphate, starch (modified or unmodified), cellulose, or any combination thereof. The free-flowing agent may be present in the composition of the first discrete unit, the composition of the second discrete unit, and/or the composition of the third discrete unit in an amount of 0.01 to 10% (w/w) g. Preferably, the free-flowing agent is present in the composition of the first discrete unit, the composition of the second discrete unit, and/or the composition of the third discrete unit in an amount of 0.1 to 8% (w/w) g. More preferably, the free-flowing agent is present in the composition of the first discrete unit, the composition of the second discrete unit, and/or the composition of the third discrete unit in an amount of 0.2 to 5% (w/w).

The composition of the first discrete unit, the composition of the second discrete unit, and the composition of the third discrete unit may be provided in the form of a powder, a gel, a suspension, a paste, a solid, a gummy, a liquid, a liquid concentrate, a reconstitutable powder, a reconstituted solution, or a ready-to-use product. Preferably, the composition is in the form of a reconstitutable powder, a liquid, a liquid concentrate, a reconstituted solution, or a ready-to-use product. Most preferably, the composition is provided in the form of a reconstitutable powder. The composition of the first discrete unit, the composition of the second discrete unit, and the composition of the third discrete unit may each be provided in the same form. Alternatively, two out of the three of the composition of the first discrete unit, the composition of the second discrete unit, and the composition of the third discrete unit may be provided in the same form. Alternatively, the composition of the first discrete unit, the composition of the second discrete unit, and the composition of the third discrete unit may each be provided in a different form.

The composition of the first discrete unit, the composition of the second discrete unit, and/or the composition of the third discrete unit may be a supplement, a nutritional composition, or a combination thereof. The nutritional composition may be a nutritional supplement, a children's nutritional product, an infant formula, a human milk fortifier, a follow-up formula, a young child milk, or any other composition designed for a paediatric subject.

The compositions of the first discrete unit, the second discrete unit, and the third discrete unit are intended for a paediatric subject. The paediatric subject may be an infant or a child. The infant may be a full-term infant or a preterm infant.

It is preferable that the composition of the first discrete unit, the composition of the second discrete unit, and/or the composition of the third discrete unit are free from other allergens (e.g. egg, wheat, soya) so that the cow's milk protein reintroduction kit is suitable for those paediatric subjects with other co-existing allergies. Alternatively, the composition of the first discrete unit, the composition of the second discrete unit, and/or the composition of the third discrete unit may contain other allergens (e.g. egg, wheat, soya).

In one preferred embodiment, the cow's milk protein reintroduction kit consists of:
at least one first discrete unit comprising a composition comprising 0.01 to 0.05 g of cow's milk protein;
at least one second discrete unit comprising a composition comprising 0.25 to 0.45 g of cow's milk protein; and,
at least one third discrete unit comprising a composition comprising 0.55 to 1 g of cow's milk protein.

In another preferred embodiment, the cow's milk protein reintroduction kit consists of:
at least one first discrete unit comprising a composition comprising 0.01 to 0.05 g of cow's milk protein and a carbohydrate source selected from the group of maltodextrin, lactose, corn syrup solids, starch, permeate, glucose solids, or any combination thereof;
at least one second discrete unit comprising a composition comprising 0.25 to 0.45 g of cow's milk protein and a carbohydrate source selected from the group of maltodextrin, lactose, corn syrup solids, starch, permeate, glucose solids, or any combination thereof; and,
at least one third discrete unit comprising a composition comprising 0.55 to 1 g of cow's milk protein and a carbohydrate source selected from the group of maltodextrin, lactose, corn syrup solids, starch, permeate, glucose solids, or any combination thereof.

In another preferred embodiment, the cow's milk protein reintroduction kit consists of:
at least one first discrete unit comprising a composition comprising 0.01 to 0.05 g of cow's milk protein, a carbohydrate source selected from the group of maltodextrin, lactose, corn syrup solids, starch, permeate, glucose solids, or any combination thereof, and a free-flowing agent;
at least one second discrete unit comprising a composition comprising 0.25 to 0.45 g of cow's milk protein, a carbohydrate source selected from the group of maltodextrin, lactose, corn syrup solids, starch, permeate, glucose solids, or any combination thereof, and a free-flowing agent; and,
at least one third discrete unit comprising a composition comprising 0.55 to 1 g of cow's milk protein, a carbohydrate source selected from the group of maltodextrin, lactose, corn syrup solids, starch, permeate, glucose solids, or any combination thereof, and a free-flowing agent.

In another preferred embodiment, the cow's milk protein reintroduction kit consists of:
at least one first discrete unit comprising a composition comprising 0.01 to 0.05 g of cow's milk protein, a carbohydrate source selected from the group of maltodextrin, lactose, corn syrup solids, starch, permeate, glucose solids, or any combination thereof, and a free-flowing agent selected from the group of tricalcium phosphate, starch (modified or unmodified), cellulose, or any combination thereof; and,
at least one second discrete unit comprising a composition comprising 0.25 to 0.45 g of cow's milk protein, a carbohydrate source selected from the group of maltodextrin, lactose, corn syrup solids, starch, permeate, glucose solids, or any combination thereof, and a free-flowing agent selected from the group of tricalcium phosphate, starch (modified or unmodified), cellulose, or any combination thereof; and,
at least one third discrete unit comprising a composition comprising 0.55 to 1 g of cow's milk protein, a carbohydrate source selected from the group of maltodextrin, lactose, corn syrup solids, starch, permeate, glucose solids, or any combination thereof, and a free-flowing agent selected from the group of tricalcium phosphate, starch (modified or unmodified), cellulose, or any combination thereof.

The features described above in relation to the first discrete unit, the second discrete unit, and the third discrete unit of the first aspect also apply to the first discrete unit, the second discrete unit, and the third discrete unit of the second, third, fourth, and fifth aspects below. The features described above in relation to the first discrete unit, the second discrete unit, and the third discrete unit of the first aspect also apply to the first discrete unit, the second discrete unit, and the third discrete unit of the sixth, seventh, eighth, ninth, and tenth aspects below, with the exception that the cow's milk protein amount is not mandatorily present in the composition of the first discrete unit, the composition of the second discrete unit, and/or the composition of the third discrete unit.

In a second aspect, the cow's milk protein reintroduction kit of the first aspect may be for use in the treatment of cow's milk allergy in a paediatric subject diagnosed with, or believed to be suffering from, cow's milk allergy.

The cow's milk protein reintroduction kit of the second aspect may be for use in the treatment of cow's milk allergy in a paediatric subject diagnosed with, or believed to be suffering from, non-IgE-mediated cow's milk allergy and/or IgE-mediated cow's milk allergy. When the cow's milk allergy is non-IgE-mediated cow's milk allergy, the non-IgE-mediated cow's milk allergy may be mild-to-moderate (i.e. non-severe) non-IgE-mediated cow's milk allergy.

In a third aspect, the cow's milk protein reintroduction kit of the first aspect may be for use in the induction of tolerance to cow's milk in a paediatric subject diagnosed with, or believed to be suffering from, cow's milk allergy.

The cow's milk protein reintroduction kit of the third aspect may be for use in the induction of tolerance to cow's milk in a paediatric subject diagnosed with, or believed to be suffering from, non-IgE-mediated cow's milk allergy and/or IgE-mediated cow's milk allergy. When the cow's milk allergy is non-IgE-mediated cow's milk allergy, the non-IgE-mediated cow's milk allergy may be mild-to-moderate (i.e. non-severe) non-IgE-mediated cow's milk allergy.

In a fourth aspect, there is provided a cow's milk protein reintroduction protocol for use in the treatment of cow's milk allergy in a paediatric subject diagnosed with, or believed to be suffering from, cow's milk allergy.

In a fifth aspect, there is provided a cow's milk protein reintroduction protocol for use in the induction of tolerance to cow's milk in a paediatric subject diagnosed with, or believed to be suffering from, cow's milk allergy.

The cow's milk protein reintroduction protocols of the fourth and fifth aspects utilise the cow's milk protein reintroduction kit of the first aspect. The cow's milk protein reintroduction protocols of the fourth and fifth aspects comprise the steps of:
1. Providing the paediatric subject with one first discrete unit for at least one day, wherein the first discrete unit comprises a composition comprising 0.01 to 0.08 g of cow's milk protein;
2. Providing the paediatric subject with two first discrete units for at least one day;
3. Providing the paediatric subject with three first discrete units for at least one day;
4. Providing the paediatric subject with one second discrete unit for at least one day, wherein the second discrete unit comprises a composition comprising 0.25 to 0.54 g of cow's milk protein;
5. Providing the paediatric subject with two second discrete units for at least one day;
6. Providing the paediatric subject with three second discrete units for at least one day;
7. Providing the paediatric subject with one third discrete unit for at least one day, wherein the third discrete unit comprises a composition comprising 0.55 to 2 g of cow's milk protein;
8. Providing the paediatric subject with two third discrete units for at least one day; and,
9. Providing the paediatric subject with three third discrete units for at least one day.

In a sixth aspect, there is provided a cow's milk protein reintroduction kit comprising:
at least one first discrete unit comprising a composition comprising 5 to 55 micrograms (μg) of Bos d 5;
at least one second discrete unit comprising a composition comprising 170 to 400 μg of Bos d 5; and,
at least one third discrete unit comprising a composition comprising at least 1250 μg of Bos d 5.

In a seventh aspect, the cow's milk protein reintroduction kit of the sixth aspect may be for use in the treatment of cow's milk allergy in a paediatric subject diagnosed with, or believed to be suffering from, cow's milk allergy.

The cow's milk protein reintroduction kit of the seventh aspect may be for use in the treatment of cow's milk allergy in a paediatric subject diagnosed with, or believed to be suffering from, non-IgE-mediated cow's milk allergy and/or IgE-mediated cow's milk allergy. When the cow's milk allergy is non-IgE-mediated cow's milk allergy, the non-IgE-mediated cow's milk allergy may be mild-to-moderate (i.e. non-severe) non-IgE-mediated cow's milk allergy.

In an eighth aspect, the cow's milk protein reintroduction kit of the sixth aspect may be for use in the induction of tolerance to cow's milk in a paediatric subject diagnosed with, or believed to be suffering from, cow's milk allergy.

The cow's milk protein reintroduction kit of the eighth aspect may be for use in the induction of tolerance to cow's milk in a paediatric subject diagnosed with, or believed to be suffering from, non-IgE-mediated cow's milk allergy and/or IgE-mediated cow's milk allergy. When the cow's milk allergy is non-IgE-mediated cow's milk allergy, the non-IgE-mediated cow's milk allergy may be mild-to-moderate (i.e. non-severe) non-IgE-mediated cow's milk allergy.

In a ninth aspect, there is provided a cow's milk protein reintroduction protocol for use in the treatment of cow's milk allergy in a paediatric subject diagnosed with, or believed to be suffering from, cow's milk allergy.

In a tenth aspect, there is provided a cow's milk protein reintroduction protocol for use in the induction of tolerance to cow's milk in a paediatric subject diagnosed with, or believed to be suffering from, cow's milk allergy.

The cow's milk protein reintroduction protocols of the ninth and tenth aspects utilise the cow's milk protein reintroduction kit of the sixth aspect. The cow's milk protein reintroduction protocols of the fourth and fifth aspects comprise the steps of:
1. Providing the paediatric subject with one first discrete unit for at least one day, wherein the first discrete unit comprises a composition comprising 5 to 55 μg of Bos d 5.
2. Providing the paediatric subject with two first discrete units for at least one day;
3. Providing the paediatric subject with three first discrete units for at least one day;
4. Providing the paediatric subject with one second discrete unit for at least one day, wherein the second discrete unit comprises a composition comprising 170 to 400 μg of Bos d 5;
5. Providing the paediatric subject with two second discrete units for at least one day;
6. Providing the paediatric subject with three second discrete units for at least one day;
7. Providing the paediatric subject with one third discrete unit for at least one day, wherein the third discrete unit comprises a composition comprising at least 1250 μg of Bos d 5;
8. Providing the paediatric subject with two third discrete units for at least one day; and,
9. Providing the paediatric subject with three third discrete units for at least one day.

As noted above, the composition of the first discrete unit, the composition of the second discrete unit, and the composition of the third discrete unit may each be subjected to different processing conditions during production, so as to yield compositions with different respective levels of allergenicity. It is therefore possible for the paediatric subject to receive a higher daily amount of cow's milk protein in steps 5 and 6 of the cow's milk protein reintroduction protocol of the fourth aspect and/or the fifth aspect, than the paediatric subject receives in steps 7 and 8. This is because the composition of the third discrete unit may be subjected to processing conditions such that the allergenicity of the composition of the third discrete unit is higher than the allergenicity of the composition of the second discrete unit and thus, the paediatric subject initially receives a lower daily amount of cow's milk protein.

The cow's milk protein reintroduction protocol of the fourth aspect, the fifth aspect, the ninth aspect, and/or the tenth aspect may be for use in the induction of tolerance to cow's milk in a paediatric subject diagnosed with, or believed to be suffering from, non-IgE-mediated cow's milk allergy and/or IgE-mediated cow's milk allergy. When the cow's milk allergy is non-IgE-mediated cow's milk allergy, the non-IgE-mediated cow's milk allergy may be mild-to-moderate (i.e. non-severe) non-IgE-mediated cow's milk allergy.
In step 1 of the cow's milk protein reintroduction protocol of the fourth aspect, the fifth aspect, the ninth aspect, and/or the tenth aspect, the one first discrete unit may be provided to the paediatric subject for one day to fourteen days, two to fourteen days, or two to seven days.
In step 2 of the cow's milk protein reintroduction protocol of the fourth aspect, the fifth aspect, the ninth aspect, and/or the tenth aspect, the two first discrete units may be provided to the paediatric subject for one day to fourteen days, two to fourteen days, or two to seven days.
In step 3 of the cow's milk protein reintroduction protocol of the fourth aspect, the fifth aspect, the ninth aspect, and/or the tenth aspect, the three first discrete units may be provided to the paediatric subject for one day to fourteen days, two to fourteen days, or two to seven days.
In step 4 of the cow's milk protein reintroduction protocol of the fourth aspect, the fifth aspect, the ninth aspect, and/or the tenth aspect, the one second discrete unit may be provided to the paediatric subject for one day to fourteen days, two to fourteen days, or two to seven days.
In step 5 of the cow's milk protein reintroduction protocol of the fourth aspect, the fifth aspect, the ninth aspect, and/or the tenth aspect, the two second discrete units may be provided to the paediatric subject for one day to fourteen days, two to fourteen days, or two to seven days.
In step 6 of the cow's milk protein reintroduction protocol of the fourth aspect, the fifth aspect, the ninth aspect, and/or the tenth aspect, the three second discrete units may be provided to the paediatric subject for one day to fourteen days, two to fourteen days, or two to seven days.
In step 7 of the cow's milk protein reintroduction protocol of the fourth aspect, the fifth aspect, the ninth aspect, and/or the tenth aspect, the one third discrete unit may be provided to the paediatric subject for one day to fourteen days, two to fourteen days, or two to seven days.
In step 8 of the cow's milk protein reintroduction protocol of the fourth aspect, the fifth aspect, the ninth aspect, and/or the tenth aspect, the two third discrete units may be provided to the paediatric subject for one day to fourteen days, two to fourteen days, or two to seven days.
In step 9 of the cow's milk protein reintroduction protocol of the fourth aspect, the fifth aspect, the ninth aspect, and/or the tenth aspect, the three third discrete units may be provided to the paediatric subject for one day to fourteen days, two to fourteen days, or two to seven days.

In respect of the second aspect, the fourth aspect, the seventh aspect, and the ninth aspect, the treatment of cow's milk allergy in a paediatric subject diagnosed with, or believed to be suffering from, IgE-mediated cow's milk allergy may refer to a reduction in the IgE level in the sera of the paediatric subject of at least 20%, compared to a control value taken prior to the initiation of the milk reintroduction process; a reduction of at least 30%; a reduction of at least 40%; a reduction of at least 50%; or, a reduction of at least 60%. A reduction in the IgE level in the sera of the paediatric subject may be quantified by the method detailed in Study 3 of the ‘Experimental Procedure’ section below, or any suitable method for determining IgE sera levels in a paediatric subject known in the art. In addition, or alternatively, the treatment of cow's milk allergy in a paediatric subject diagnosed with, or believed to be suffering from, IgE-mediated cow's milk allergy may refer to an increase in the IgG4 level in the sera of the paediatric subject of at least 10%, compared to a control value taken prior to the initiation of the milk reintroduction process; an increase of at least 20%; an increase of at least 30%; an increase of at least 40%; or, an increase of at least 50%. An increase in the IgG4 level in the sera of the paediatric subject may be quantified by the method detailed in Study 3 of the ‘Experimental Procedure’ section below, or any suitable method for determining IgG4 sera levels in a paediatric subject known in the art.

In respect of the third aspect, the fifth aspect, the eighth aspect, and the tenth aspect, the induction of tolerance to cow's milk in a paediatric subject diagnosed with, or believed to be suffering from, IgE-mediated cow's milk allergy may refer to a reduction in the IgE level in the sera of the paediatric subject of at least 20%, compared to a control value taken prior to the initiation of the milk reintroduction process; a reduction of at least 30%; a reduction of at least 40%; a reduction of at least 50%; or, a reduction of at least 60%. A reduction in the IgE level in the sera of the paediatric subject may be quantified by the method detailed in Study 3 of the ‘Experimental Procedure’ section below, or any suitable method for determining IgE sera levels in a paediatric subject known in the art. In addition, or alternatively, the induction of tolerance to cow's milk in a paediatric subject diagnosed with, or believed to be suffering from, IgE-mediated cow's milk allergy may refer to an increase in the IgG4 level in the sera of the paediatric subject of at least 10%, compared to a control value taken prior to the initiation of the milk reintroduction process;

an increase of at least 20%; an increase of at least 30%; an increase of at least 40%; or, an increase of at least 50%. An increase in the IgG4 level in the sera of the paediatric subject may be quantified by the method detailed in Study 3 of the ‘Experimental Procedure’ section below, or any suitable method for determining IgG4 sera levels in a paediatric subject known in the art.

The scope of the present invention is defined in the appended claims. It is to be understood that the skilled person may make amendments to the scope of the claims without departing from the scope of the present disclosure.

### EXAMPLES

### Example 1: Example Guidance as to the Use of the Cow's Milk Protein Reintroduction Kit and the Cow's Milk Protein Reintroduction Protocol

Prior to employing the cow's milk protein reintroduction kit/cow's milk protein reintroduction protocol, the paediatric subject diagnosed with, or believed to be suffering from, non-IgE-mediated cow's milk allergy or IgE-mediated cow's milk allergy should ideally have eliminated cow's milk from their diet i.e. begun an elimination diet. The elimination diet should ideally last for up to four weeks, but certainly for a minimum of two weeks, to ensure a clear improvement of symptoms, if not a complete resolution.

All paediatric subjects who have been diagnosed with, or are suspected of having, non-IgE-mediated cow's milk allergy or IgE-mediated cow's milk allergy should start with the first discrete unit. To begin with, the paediatric subject is provided one first discrete unit per day (i.e. the lowest cow's milk protein amount), for at least one day. The number of days that the paediatric subject receives each particular cow's milk protein amount (i.e. the particular step of the cow's milk protein reintroduction protocol), before moving onto the next cow's milk protein amount (i.e. the next step of the cow's milk protein reintroduction protocol), is based on the characteristics of each individual paediatric subject.

If the addition of the one first discrete unit per day to the paediatric subject's diet does not cause any clinically-relevant symptoms, which may be one or more of the symptoms detailed in Table 1, to reappear, or if the addition of the one first discrete unit per day to the paediatric subject's diet does cause any clinically-relevant symptoms to reappear but these clinically-relevant symptoms decrease in the number and/or severity over the course of a number of days of the paediatric subject receiving the first discrete unit, ideally to no clinically-relevant symptoms, then the paediatric subject is deemed to have ‘tolerated’ the lowest amount of cow's milk protein and can then move onto receiving two first discrete units per day (i.e. the second step of the cow's milk protein reintroduction protocol), for at least one day. If the addition of the one first discrete unit per day to the paediatric subject's diet does cause any clinically-relevant symptoms to reappear, which do not decrease in the number and/or severity over the course of a number of days of the paediatric subject receiving the first discrete unit, it is suggested that the paediatric subject stop the cow's milk protein reintroduction kit/protocol and that the cow's milk protein reintroduction kit/protocol is repeated in approximately four to six months' time.

TABLE 1
National Institute for Health and Care Excellence (NICE) guidance
as to signs and symptoms of possible food allergy in under 19s

| Area of the Body | IgE-Mediated | Non IgE-Mediated |
|---|---|---|
| Skin | Pruritus | Pruritus |
| | Erythema | Erythema |
| | Acute urticaria (localised or | Atopic eczema |
| | generalized) | |
| | Acute angioedema (lips, | |
| | face, and/or around the eyes | |
| Gastrointestinal | Angioedema (lips, tongue, | Gastro-oesophageal reflux |
| system | and/or palate) | disease |
| | Oral pruritus | Loose or frequent stools |
| | Nausea | Blood and/or mucus in stools |
| | Colicky abdominal pain | Abdominal pain |
| | Vomiting | Infantile colic |
| | Diarrhoea | Food refusal or aversion |
| | | Constipation |
| | | Perianal redness |
| | | Pallor and tiredness |
| | | Faltering growth, along with |
| | | gastrointestinal symptoms |
| | | (with/without significant atopic |
| | | eczema) |
| Respiratory system | Lower respiratory tract symptoms (cough, chest tightness, | |
| | wheezing or shortness of breath) | |
| | Upper respiratory tract | |
| | symptoms (nasal itching, | |
| | sneezing, rhinorrhoea, or | |
| | congestion (with/without | |
| | conjunctivitis) | |
| Other | Signs or symptoms of | |
| | anaphylaxis or other | |
| | systemic allergic reactions | |

If the addition of the two first discrete units per day to the paediatric subject's usual diet does not cause any clinically-relevant symptoms to reappear, or if the addition of the two first discrete units per day to the paediatric subject's diet does cause any clinically-relevant symptoms to reappear but these clinically-relevant symptoms decrease in the number and/or severity over the course of a number of days of the paediatric subject receiving the first discrete unit, ideally to no clinically-relevant symptoms, then the paediatric subject is deemed to have ‘tolerated’ the particular amount of cow's milk protein and can then move onto receiving three first discrete units per day, for at least one day, and so on. If a particular, higher cow's milk protein amount (e.g. two second discrete units per day, for at least one day i.e. step five of the cow's milk protein reintroduction protocol) does cause clinically-relevant symptoms to reappear, which do not decrease in the number and/or severity over the course of a number of days of the paediatric subject receiving the particular cow's milk protein amount, it is suggested that the paediatric subject should go back to the previous cow's milk protein amount (in this instance, step four of the cow's milk protein reintroduction protocol) and continue with this cow's milk protein amount for a more prolonged period than previously, before again trying the higher cow's milk protein amount that initially caused the clinically-relevant symptoms to reappear (in this instance, step five of the cow's milk protein reintroduction protocol).

If the paediatric subject successfully tolerates three third discrete units per day, for at least one day (i.e. step nine of the cow's milk protein reintroduction protocol), preferably two to fourteen days, then the paediatric subject should move on to other cow's milk-containing food products e.g. first cheese, then yoghurt, and finally, pasteurised milk and/or cow's milk-based paediatric formula, under the guidance of a health care professional.

### DESCRIPTION OF FIGURES

FIG. 1: (a) A plot showing the amount of Bos d 5 in a baked cookie and half of a baked muffin i.e. in a serving of each; (b) A plot showing the amount of Bos d 5 in a serving of liquid skimmed (non-fat) milk.

### EXPERIMENTAL PROCEDURE

### Study 1: Determination of Bos d 5 Levels

Baked cookies was prepared by adding skimmed (non-fat) dairy milk powder to other conventional cookie ingredients, obtained from a local supermarket, and cooked in a domestic kitchen by one of the inventors. The unbaked cookies were baked at 177° C. (350° F.) for 15 minutes. A baked muffin was prepared by adding liquid cow's milk to other conventional muffin ingredients, obtained from a local supermarket, and cooked in a domestic kitchen by one of the inventors. The unbaked muffins were baked at 177° C. (350° F.) for 20 minutes.

The Bos d 5 levels in cow's milk-containing foods (μg/g) could be determined by an enzyme-linked immunosorbent assay (ELISA) specific for Bos d 5, as detailed in Hindley et al. (Clin. Exp. Allergy, 2021), which is briefly summarised below.

In Hindley et al., a two-site monoclonal antibody (mAb) ELISA was used to determine Bos d 5 levels in a muffin, which utilised mAb NBD5-1 for allergen capture and biotinylated mAb NBD5-2 for detection. Purified Bos d 5 was used as the assay standard for the ELISA.

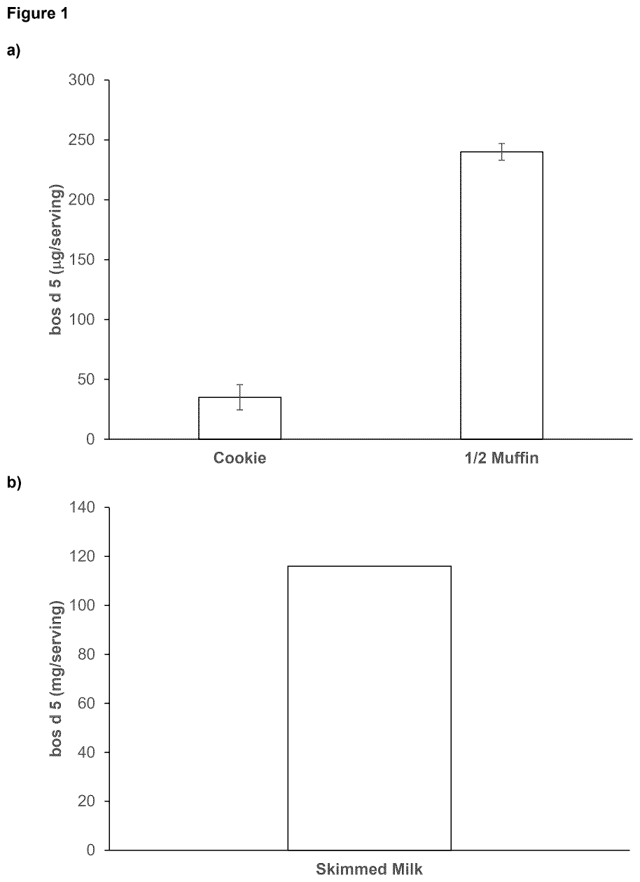

As shown in FIGS. 1(a) and 1(b), the amount of Bos d 5 increases from a baked cookie to half of a baked muffin, and onto liquid skimmed (non-fat) milk. The determined Bos d 5 amounts in the cow's milk-containing foods have been utilised as a guide for the desired Bos d 5 amounts in the discrete units of the defined cow's milk protein reintroduction kit and cow's milk protein reintroduction protocol.

Study 2: Evaluating the Efficacy of the Cow's Milk Protein Reintroduction Kit and the Cow's Milk Protein Reintroduction Protocol in a Paediatric Subject Diagnosed with, or Believed to be Suffering from, Non-IgE-Mediated Cow's Milk Allergy

The treatment of non-IgE-mediated cow's milk allergy and/or the induction of tolerance to cow's milk in a paediatric subject involves a milk rechallenge. The cow's milk protein reintroduction kit/protocol of the present invention constitutes the important initial stages of a milk rechallenge. The cow's milk protein reintroduction kit/protocol of the present invention should be followed by the sequential introduction of cheese, yoghurt, and pasteurised milk and/or cow's milk-based paediatric formula, into the paediatric subject's diet, under the guidance of a health care professional.

There is currently no general consensus as to the exact physiological processes underlying a clinical manifestation of non-IgE-mediated CMA. Thus, for non-IgE-mediated CMA, there isn't a generally-accepted physiological parameter that can be measured, during a milk rechallenge in a paediatric subject, in order to determine the efficacy of the treatment of non-IgE-mediated CMA and/or the induction of tolerance to cow's milk.

It is for this reason that, at present, the most suitable method of determining the efficacy of the treatment of non-IgE-mediated CMA and/or induction of tolerance to cow's milk, during the milk rechallenge, is to monitor the clinically-relevant symptoms presented by the paediatric subject.

For non-IgE-mediated CMA, the milk rechallenge should ideally last for a minimum of a week, with treatment of non-IgE-mediated CMA being the observation of a decrease in the number and/or severity of clinically-relevant symptoms as the paediatric subject progresses through the milk rechallenge. Tolerance to cow's milk is deemed to be attained when no clinically-relevant symptoms are observed at the completion of the milk rechallenge. If tolerance to cow's milk is deemed to be achieved, the milk rechallenge should still be continued for a period of time (ideally at least a month), to truly indicate tolerance development.

In the near future, it is possible that experiments such as a basophil activation test, a T-cell functionality test, a transcriptomics-related test, an epigenetics-related test, a cytokine profile test, or similar, may be suitable methods for specifically quantifying the treatment of non-IgE-mediated CMA and/or induction of tolerance to cow's milk, before, during, and after a milk rechallenge.

Study 3: Evaluating the Efficacy of the Cow's Milk Protein Reintroduction Kit and the Cow's Milk Protein Reintroduction Protocol in a Paediatric Subject Diagnosed with, or Believed to be Suffering from IgE-Mediated Cow's Milk Allergy

The treatment of IgE-mediated CMA and/or the induction of tolerance to cow's milk in a paediatric subject involves a milk rechallenge. The cow's milk protein reintroduction kit/protocol of the present invention constitutes the important initial stages of a milk rechallenge. The cow's milk protein reintroduction kit/protocol of the present invention should be followed by the sequential introduction of cheese, yoghurt, and pasteurised milk and/or cow's milk-based paediatric formula, into the paediatric subject's diet, under the guidance of a health care professional. For IgE-mediated CMA, the milk rechallenge should last for a minimum of a day.

As with non-IgE-mediated CMA, the treatment of IgE-mediated CMA may be determined by the observation of a decrease in the number and/or severity of clinically-relevant symptoms as the paediatric subject progresses through the milk rechallenge. Tolerance to cow's milk is deemed to be attained when no clinically-relevant symptoms are observed at the completion of the milk rechallenge. If tolerance to cow's milk is deemed to be achieved, the milk rechallenge should still be continued for a period of time (ideally at least a month), to truly indicate tolerance development.

However, with IgE-mediated CMA, it may be possible to quantitatively measure the efficacy of the treatment of IgE-mediated CMA and/or the induction of tolerance to cow's milk, in the paediatric subject, during the milk rechallenge. A suitable method of quantitative measurement may involve measuring the immunoglobulin E (IgE) and/or immunoglobulin G4 (IgG4) levels in the serum of the paediatric subject e.g. using ImmunoCAPT allergen-specific IgE and/or IgG4 tests (ThermoFisher Scientific). The level of IgE and/or IgG4 in the serum of the paediatric subject would be determined prior to the commencement of the milk rechallenge (i.e. the control/baseline value), with regular measurements taken throughout the milk rechallenge. The treatment of IgE-mediated CMA and/or the induction of tolerance to cow's milk may be shown by reduction in the IgE levels and/or increase in IgG4 levels. Another suitable method of quantitative measurement may involve a cellular assay, such as a basophil activation test. The basophil activation test is a flow cytometry-based assay, which involves a sample of the subject's blood, and measures the expression of activation markers on the surface of basophils. In a basophil activation test, the treatment of IgE-mediated CMA and/or the induction of tolerance to cow's milk may be shown by decreased basophil reactivity and a lower ratio of milk-specific basophil reactivity to non-specific basophil activation, compared to cow's milk-reactive individuals (Ford et al., J. Allergy Clin. Immunol., 2012).

1. A cow's milk protein reintroduction kit comprising:
at least one first discrete unit comprising a composition comprising 0.01 to 0.08 g of cow's milk protein;
at least one second discrete unit comprising a composition comprising 0.25 to 0.54 g of cow's milk protein; and,
at least one third discrete unit comprising a composition comprising 0.55 to 2 g of cow's milk protein.

2. The cow's milk protein reintroduction kit of claim 1, wherein the first discrete unit, the second discrete unit and/or the third discrete unit further comprises a carbohydrate source.

3. The cow's milk protein reintroduction kit of claim 1, wherein the 0.01 to 0.08 g of cow's milk protein of first discrete unit comprises 5 to 55 micrograms (μg) of Bos d 5, the 0.25 to 0.54 g of cow's milk protein of the second discrete unit comprises 170 to 400 μg of Bos d 5, and the 0.55 to 2 g of cow's milk protein of the third discrete unit comprises at least 1250 μg of Bos d 5.

4. The cow's milk protein reintroduction kit of claim 1, wherein the at least one first discrete unit comprises a composition comprising 0.01 to 0.07 g of cow's milk protein.

5. The cow's milk protein reintroduction kit of claim 1, wherein the at least one first discrete unit comprises two to one hundred first discrete units.

6. (canceled)

7. The cow's milk protein reintroduction kit of claim 1, wherein the at least one second discrete unit comprises a composition comprising 0.25 to 0.5 g of cow's milk protein.

8. The cow's milk protein reintroduction kit of claim 1, wherein the at least one second discrete unit comprises two to one hundred second discrete units.

9. (canceled)

10. The cow's milk protein reintroduction kit of claim 1, wherein the at least one third discrete unit comprises a composition comprising 0.55 to 1.5 g of cow's milk protein.

11. (canceled)

12. The cow's milk protein reintroduction kit of claim 1, wherein the at least one third discrete unit comprises two to one hundred third discrete units.

13. The cow's milk protein reintroduction kit of claim 1, wherein the composition of the first discrete unit, the composition of the second discrete unit, and/or the composition of the third discrete unit is a supplement or a nutritional composition.

14. The cow's milk protein reintroduction kit of claim 1, wherein the composition of the first discrete unit, the composition of the second discrete unit, and/or the composition of the third discrete unit is a synthetic composition.

15-25. (canceled)

26. A cow's milk protein reintroduction kit comprising:
at least one first discrete unit comprising a composition comprising 5 to 55 micrograms (μg) of Bos d 5;
at least one second discrete unit comprising a composition comprising 170 to 400 μg of Bos d 5; and,
at least one third discrete unit comprising a composition comprising at least 1250 μg of Bos d 5.

27. The cow's milk protein reintroduction kit of claim 26, wherein the first discrete unit, the second discrete unit, and/or the third discrete unit further comprises a carbohydrate source.

28. The cow's milk protein reintroduction kit of claim 26, wherein the first discrete unit comprises a composition comprising 15 to 55 μg of Bos d 5.

29-30. (canceled)

31. The cow's milk protein reintroduction kit of claim 26, wherein the second discrete unit comprises a composition comprising 200 to 350 μg of Bos d 5.

32-33. (canceled)

34. The cow's milk protein reintroduction kit of claim 26, wherein the third discrete unit comprises a composition comprising 1250 μg to 75 milligrams (mg) of Bos d 5.

35-36. (canceled)

37. The cow's milk protein reintroduction kit of claim 26, wherein the composition of the first discrete unit, the composition of the second discrete unit, and/or the composition of the third discrete unit is a supplement or a nutritional composition.

38. The cow's milk protein reintroduction kit of claim 26, wherein the composition of the first discrete unit, the composition of the second discrete unit, and/or the composition of the third discrete unit is a synthetic composition.

39-48. (canceled)

49. A method of treating cow's milk allergy and/or inducing tolerance to cow's milk in a paediatric subject diagnosed with, or believed to be suffering from, cow's milk allergy comprising administering to the paediatric subject a cow's milk protein reintroduction kit comprising:
at least one first discrete unit comprising a composition comprising 0.01 to 0.08 g of cow's milk protein;
at least one second discrete unit comprising a composition comprising 0.25 to 0.54 g of cow's milk protein; and,
at least one third discrete unit comprising a composition comprising 0.55 to 2 g of cow's milk protein.

50. The method of treating cow's milk allergy and/or inducing tolerance to cow's milk in a paediatric subject diagnosed with, or believed to be suffering from, cow's milk allergy of claim 49, wherein the cow's milk allergy is non-IgE-mediated cow's milk allergy.

51. The method of treating cow's milk allergy and/or inducing tolerance to cow's milk in a paediatric subject diagnosed with, or believed to be suffering from, cow's milk allergy of claim 49, wherein the cow's milk allergy is IgE-mediated cow's milk allergy.
